(19) United States
(12) Patent Application Publication
(10) Pub. No.: US 2016/0089363 A1
(43) Pub. Date: Mar. 31, 2016

(54) COMPOSITIONS AND METHODS FOR TREATING MICROBIOTA-RELATED PSYCHOTROPIC CONDITIONS AND DISEASES

(71) Applicant: Thomas Julius BORODY, Five Dock (AU)

(72) Inventor: Thomas Julius BORODY, Five Dock (AU)

(21) Appl. No.: 14/888,081

(22) PCT Filed: Apr. 30, 2014
(86) PCT No.: PCT/AU2014/000478

§ 371 Date: Oct. 29, 2015

Related U.S. Application Data
(60) Provisional application No. 61/817,769, filed on Apr. 30, 2013.

(51) Int. Cl. A61K 31/437; A61K 45/06; A61K 31/7036; A61K 38/14; A61K 31/427

(52) U.S. Cl. CPC A61K 31/437; A61K 38/14; A61K 31/427; A61K 31/7036; A61K 45/06

(57) ABSTRACT

In alternative embodiments, the invention provides compositions and methods for treating, ameliorating and preventing various disorders and conditions in mammals, including genetically-predisposed and chronic disorders, where the microbial or bacterial flora of the bowel is at least one causative or symptom-producing factor, for example, where the microbial or bacterial flora of the bowel manufactures neurotoxins or neurotoxic agents that enter the body through the gastrointestinal (GI) tract, e.g. the colon, and reach the systemic space, e.g., by neural streaming or via the circulation, to reach the central nervous system (CNS), including the brain, the peripheral nervous system (PNS), and other nervous systems. In alternative embodiments, methods and compositions of the invention comprise or comprise use of medications, formulations and pharmaceuticals comprising rifaximin or equivalent active agents that can suppress or eradicate the microbiota super-infection that causes various psychotropic disorders. These compositions have been found to be affective in a broad spectrum of disorders but particularly in the obsessive compulsive disorder group (OCD).

### TECHNICAL FIELD

This invention generally relates to medicine and gastroenterology, pharmacology and microbiology. In alternative embodiments, the invention provides compositions and methods for treating, ameliorating and preventing various disorders and conditions in mammals, including genetically-predisposed and chronic disorders, where the microbial or bacterial flora of the bowel is at least one causative or symptom-producing factor, for example, where the microbial or bacterial flora of the bowel manufactures neurotoxins or neurotoxic agents that enter the body through the gastrointestinal (GI) tract, e.g. the colon, and reach the systemic space, e.g., by neural streaming or via the circulation, to reach the central nervous system (CNS), including the brain, the peripheral nervous system (PNS), and other nervous systems. In alternative embodiments, methods and compositions of the invention comprise or comprise use of medications, formulations and pharmaceuticals comprising active agents that can suppress or eradicate the microbiota super-infection that causes various psychotropic disorders. These compositions have been found to be affective in a broad spectrum of disorders but particularly in the obsessive compulsive disorder group (OCD).

### BACKGROUND

Until recent years most mental disorders including obsessive compulsive disorder (OCD) have been, and continue to be, considered as being caused by psychological or psychiatric aberrations. Sigmund Freud indicated that OCD behaviour was due to unconscious conflicts that later manifested as symptoms. He reached back into early childhood experiences where the ‘need to touch’ which was externally prohibited by parents created an OCD type behaviour. Most OCD therapy is currently psychological therapy, but it can also use drugs to suppress certain neurotransmitter activity within the brain.

Obsessive compulsive disorders are classified under ‘anxiety disorders’ where the patient has a thinking process which produces uneasiness, apprehension, worry, fear, repetitive behaviours all aimed at reducing the anxiety. The obsessions can be associated with compulsions. The symptoms can include repetitive religious thoughts, aversion to particular numbers, nervous rituals, opening closing doors, excessive washing or cleaning, repeated checking, extreme hoarding movements of arms or legs or entering and leaving a room. These symptoms interfere with life and can alienate friends and relatives. They can cause severe emotional and financial distress. However, OCD sufferers do recognize their obsessions and compulsions and they realise they are irrational. This further distresses them. More than 80% of these begin in childhood and there is a certain amount of cross over between OCD and other disorders which share such behaviour, including autism spectrum disorder, ADHD, PTSD and profound anxiety.

Patients with OCD do not have below-average intelligence and OCD is actually associated with a higher IQ. OCD syndrome can also co-exist with major depressive disorders, bipolar disorders, anorexia nervosa, bulimia, generalised anxiety disorder, Tourrets' syndrome, Asperger's syndrome, Attention Deficit Hyperactivity Disorder. Dermatillomania, Trichotillomania as well as body dismorphic disorder can be associated with OCD. Delayed sleep may be also a feature but particularly depression is an extremely common syndrome among OCD patients. In around 80% of the cases symptoms present before the age of 18 with 1 to 3% of the population suffering with this disorder. It is estimated that in the US lifetime prevalence of OCD is 2.5%. Some estimates also say that around 2.2 million US residents have or have had OCD. Other countries have similar rates.

Patients with this disability with its associated co-morbidities, are therefore looking for some advance in therapy rather than continuing with the partially-effective but never curative psychological intervention such as behavioural and cognitive/behavioural therapies. Pharmacological treatment can lead to substantial reduction in symptoms but they do keep on persisting at moderate levels throughout life. To be completely symptom free is uncommon.

Children with autism have been noted to possess abnormal Clostridia and the Desulfovibrio bacteria in the stool, see e.g., Finegold (2012) Anaerobe 18:260.

### SUMMARY

In alternative embodiments, the invention provides formulations, pharmaceuticals or pharmaceutical preparations comprising at least one active agent, wherein the active agent comprises:
(a) a rifaximin, an extended intestinal release (EIR) rifaximin, a rifamycin derivative, a rifampicin (or rifampin), a rifabutin, a rifapentine, a rifalazil, a bicozamycin, a XIFAXAN™ (Salix Pharmaceuticals), or a mixture or combination thereof,
(b) an aminoglycoside, a gentamycin, a neomycin, a streptomycin, a paromomycin, a verdamicin, a mutamicin, a sisomicin, a netilmicin, a retymicin and/or a kanamycin, or a mixture or combination thereof,
(c) an aztreonam, a AZACTAM™, a CAYSTON™, an aztreonam macrolide, a clarithromycin (a TRUCLAR™, CRIXAN™, CLARITT™, CLARAC™, BIAXIN™, KLARICID™, KLACID™, KLARAM™, KLABAX™, CLARIPEN™, CLAREM™, CLARIDAR™, FROMILID™, CLACID™, CLACEE™, VIKROL™, INFEX™, CLARIWIN™, RESCLAR™, RANBAXY™ or a CLARIHEXAL™), a dirithromycin (a DYNABAC™), a roxithromycin (a XTHROCIN™, ROXL-150™, ROXO™, SURLID™), a telithromycin (a KETEK™), an azithromycin, a ZITHROMAX™, a AZITHROCIN™), or a mixture or combination thereof,
(d) a bismuth antibiotic, a bismuth subsalicylate, a PEPTO-BISMOL™, a PINK BISMUTH™, or a mixture or combination thereof,
(e) a vancomycin, a streptomycin, a fidaxomicin, a gentamicin, a kanamycin, an amikacin, an arbekacin, a neomycin, a netilmicin, a paromomycin, rhodostreptomycin, a tobramycin, an apramycin, or a mixture or combination thereof,
(f) a mixture or a combination of any one or several of (a), (b), (c), (d) or (e);
wherein the formulation, a pharmaceutical or a pharmaceutical preparation is formulated as a chewable delivery vehicle, a gum, a gummy, a candy, a lozenge, an ice cream or an ice, or a yogurt,
and optionally the unit dosage is a pediatric unit dosage, and optionally the unit dosage is between about 10 mg and 1100 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000 or 1100 or more mg per unit dose,
and optionally the daily dosage is about 100, 125, 150, 175, 200, 225, 250, 275, 300, 325, 350, 375, 400, 425, 450, 475, 500, 600, 700, 750, 800, 900, 1000 or 1100 or more mg per day, or between about 100 and 1100 mgm per day,
or optionally the unit dosage is set for bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day (so five times a day for adult use, or for a 70 kg individual would be 200 mg per unit dose); or for a pediatric dosage about 350 mg/25 kg a day, or about 15 to 16 mg/kg, a day; or equivalent (so five times a day for pediatric use, or for a 25 kg individual, would be 70 mg per unit dose),
and optionally the formulation, pharmaceutical or pharmaceutical preparation comprises a combination of a rifaximin together with a vancomycin.

In alternative embodiments, the formulation, pharmaceutical or pharmaceutical preparation further comprises: a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof. In alternative embodiments, the formulation, pharmaceutical or pharmaceutical preparation further comprises: a preservative, a benzoic acid, a potassium sorbate.

In alternative embodiments, the formulation, pharmaceutical or pharmaceutical preparation further comprises or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E coli, a Strep fecalis and equivalents.

In alternative embodiments, the formulation, pharmaceutical or pharmaceutical preparation further comprises or has added to: at least one congealing agent, wherein optionally the congealing agent comprises an arrowroot or a plant starch, a powdered flour, a powdered potato or potato starch, an absorbant polymer, an Absorbable Modified Polymer (AMP®), EndoClot, Santa Clara, Calif.), and/or a corn flour or a corn starch.

In alternative embodiments, the formulation, pharmaceutical or pharmaceutical preparation further comprises or has added to: at least one an anti-inflammatory agent, wherein optionally the inflammatory agent comprises or is a 4 or a 5-amino-salicylate, an olsalazine (e.g., DIPENTUM™), a mesalazine (also known as mesalamine or a 5-aminosalicylic acid (5-ASA), e.g., ASACOL™ or LIALDA™), a sulfasalazine (e.g., AZULFIDINE™, SALAZOPYRIN™ or SULAZINE™), and/or a balsalazide (e.g. COLAZAL™ or COLAZIDE™), or an equivalent thereof or a combination thereof.

In alternative embodiments, the formulation, pharmaceutical or pharmaceutical preparation further comprises or has added to: an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, and/or a prebiotic nutrient.

In alternative embodiments, the formulation, pharmaceutical or pharmaceutical preparation further comprises or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

In alternative embodiments, the formulation, pharmaceutical or pharmaceutical preparation is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, such as EUDRAGIT S™ (Evonik Industries AG, Essen, Germany), which dissolves at pH 7 or greater, e.g., comprises a multimatrix (MMX) formulation.

In alternative embodiments, the formulation, pharmaceutical or pharmaceutical preparation further comprises or has added to: an additional antimicrobial or antibiotic, wherein optionally the additional antimicrobial or antibiotic comprises: an ampicillin, a sulbactama tetracycline, a cephalosporin, a carbapenem, an imipenem, a meropenem, a MONAN™, MERONEM™, a monobactam, a lincosamide, a clindamycin, a DALACIN™, a quinolone, a fluoroquinolone, a sulphonamide, a fradicin, a NEOBIOTIC™, a nitroimidazole, a metronidazole, a tinidazole, an anti-clostridial agent, or a ramoplanan, an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin, a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or an equivalent thereof or a combination thereof.

In alternative embodiments, the invention provides a delivery vehicle, a product of manufacture, a container, a syringe, a device or a bag or device, comprising: a formulation, a pharmaceutical or a pharmaceutical preparation of the invention.

In alternative embodiments, the invention provides a delivery vehicle, a product of manufacture, a container, a syringe, a device or a bag or device comprising: a formulation, a pharmaceutical or a pharmaceutical preparation of the invention, initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In alternative embodiments, the invention provides methods for the treating, ameliorating and preventing obsessive compulsive disorder group (OCD) psychotropic disorders and conditions, an Attention Deficit Disorder (ADD and ADHD), an obsessive compulsive disorder (OCD), a depression, a schizophrenia and/or a mood disorder, or a hepatic encephalopathy, a hepatic encephalopathy, or a depressive disorder, a bipolar disorder, an anorexia nervosa, a bulimia, a generalised anxiety disorder, a Tourrets' syndrome, Asperger's syndrome or Attention Deficit Hyperactivity Disorder, comprising administering to an individual in need thereof:
(1) a formulation, a pharmaceutical or a pharmaceutical preparation of the invention; or
(2) a formulation, a pharmaceutical or a pharmaceutical preparation comprising at least one active agent, wherein the active agent comprises:
   (a) a rifaximin, an extended intestinal release (EIR) rifaximin, a rifamycin derivative, a rifampicin (or rifampin), a rifabutin, a rifapentine, a rifalazil, a bicozamycin, a XIFAXAN™ (Salix Pharmaceuticals), or a mixture or combination thereof,
   (b) an aminoglycoside, a gentamycin, a neomycin, a streptomycin, a paromomycin, a verdamicin, a mutamicin, a sisomicin, a netilmicin, a retymicin and/or a kanamycin, or a mixture or combination thereof,
   (c) an aztreonam, a AZACTAM™, a CAYSTON™, an aztreonam macrolide a clarithromycin (a TRUCLAR™, CRIXAN™, CLARITT™, CLARAC™, BIAXIN™, KLARICID™, KLACID™, KLARAM™, KLABAX™, CLARIPEN™, CLAREM™, CLARIDAR™, FROMILID™, CLACID™, CLACEE™, VIKROL™, INFEX™, CLARIWIN™, RESCLAR™, RANBAXY™ or a CLARIHEXAL™), a dirithromycin (a DYNABAC™), a roxithromycin (a XTHROCIN™, ROXL-150™, ROXO™, SURLID™), a telithromycin (a KETEK™), an azithromycin, a ZITHROMAX™, a AZITHROCIN™), or a mixture or combination thereof,
   (d) a bismuth antibiotic, a bismuth subsalicylate, a PEPTO-BISMOL™, a PINK BISMUTH™, or a mixture or combination thereof,
   (e) a vancomycin, a streptomycin, a fidaxomicin, a gentamicin, a kanamycin, an amikacin, an arbekacin, a neomycin, a netilmicin, a paromomycin, rhodostreptomycin, a tobramycin, an apramycin, or a mixture or combination thereof,
   (f) a mixture or a combination of any one or several of (a), (b), (c), (d) or (e); or
   (c) a delivery vehicle, a product of manufacture, a container, a syringe, a device or a bag of the invention, or a delivery vehicle, a product of manufacture, a container, a syringe, a device or a bag comprising one or more active agent or composition of (b);
wherein optionally the formulation, a pharmaceutical or a pharmaceutical preparation is formulated as a chewable delivery vehicle, a gum, a gummy, a candy, a lozenge, an ice cream or an ice, or a yogurt,
and optionally the unit dosage is a pediatric unit dosage, and optionally the unit dosage is between about 10 mg and 1000 mgm, or is about 10, 20, 30, 40, 50, 60, 70, 75, 80, 90, 100, 125, 150, 175, 200, 225, 250, 275, 300, 325, 350, 375, 400, 425, 450, 475 or 500 or more mg per unit dose,
or optionally the unit dosage is set for bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day (so five times a day for adult use, or for a 70 kg individual would be 200 mg per unit dose); or for a pediatric dosage about 350 mg/25 kg a day, or about 15 to 16 mg/kg, a day; or equivalent (so five times a day for pediatric use, or for a 25 kg individual, would be 70 mg per unit dose).

In alternative embodiments, the invention provides uses of:
(1) a formulation, a pharmaceutical or a pharmaceutical preparation of the invention; or
(2) a formulation, a pharmaceutical or a pharmaceutical preparation comprising at least one active agent, wherein the active agent comprises:
   (a) a rifaximin, an extended intestinal release (EIR) rifaximin, a rifamycin derivative, a rifampicin (or rifampin), a rifabutin, a rifapentine, a rifalazil, a bicozamycin, a XIFAXAN™ (Salix Pharmaceuticals), or a mixture or combination thereof,
   (b) an aminoglycoside, a gentamycin, a neomycin, a streptomycin, a paromomycin, a verdamicin, a mutamicin, a sisomicin, a netilmicin, a retymicin and/or a kanamycin, or a mixture or combination thereof,
   (c) an aztreonam, a AZACTAM™, a CAYSTON™, an aztreonam macrolide a clarithromycin (a TRUCLAR™, CRIXAN™, CLARITT™, CLARAC™, BIAXIN™, KLARICID™, KLACID™, KLARAM™, KLABAX™, CLARIPEN™, CLAREM™, CLARIDAR™, FROMILID™, CLACID™, CLACEE™, VIKROL™, INFEX™, CLARIWIN™, RESCLAR™, RANBAXY™ or a CLARIHEXAL™), a dirithromycin (a DYNABAC™), a roxithromycin (a XTHROCIN™, ROXL-150™, ROXO™, SURLID™), a telithromycin (a KETEK™), an azithromycin, a ZITHROMAX™, a AZITHROCIN™), or a mixture or combination thereof,
   (d) a bismuth antibiotic, a bismuth subsalicylate, a PEPTO-BISMOL™, a PINK BISMUTH™, or a mixture or combination thereof,
   (e) a vancomycin, a streptomycin, a fidaxomicin, a gentamicin, a kanamycin, an amikacin, an arbekacin, a neomycin, a netilmicin, a paromomycin, rhodostreptomycin, a tobramycin, an apramycin, or a mixture or combination thereof,
   (f) a mixture or a combination of any one or several of (a), (b), (c), (d) or (e); or
(3) a delivery vehicle, a product of manufacture, a container, a syringe, a device or a bag of the invention, or a delivery vehicle, a product of manufacture, a container, a syringe, a device or a bag comprising at least one active agent of (b);
in the preparation of a medicament for treating, ameliorating and preventing obsessive compulsive disorder group (OCD) psychotropic disorders and conditions, an Attention Deficit Disorder (ADD and ADHD), an obsessive compulsive disorder (OCD), a depression, a schizophrenia and/or a mood disorder, or a hepatic encephalopathy, a hepatic encephalopathy, or a depressive disorder, a bipolar disorder, an anorexia nervosa, a bulimia, a generalised anxiety disorder, a Tourrets' syndrome, Asperger's syndrome or Attention Deficit Hyperactivity Disorder, comprising administering to an individual in need thereof, or
for treating, ameliorating and preventing obsessive compulsive disorder group (OCD) psychotropic disorders and conditions, an Attention Deficit Disorder (ADD and ADHD), an obsessive compulsive disorder (OCD), a depression, a schizophrenia and/or a mood disorder, or a hepatic encephalopathy, a hepatic encephalopathy, or a depressive disorder, a bipolar disorder, an anorexia nervosa, a bulimia, a generalised anxiety disorder, a Tourrets' syndrome, Asperger's syndrome or Attention Deficit Hyperactivity Disorder, comprising administering to an individual in need thereof.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, the invention provides compositions, e.g., formulations and pharmaceutical preparations, products of manufacture, and containers and delivery vehicles, and devices and delivery materials, for treating, ameliorating and preventing various disorders and conditions in mammals, including genetically-predisposed and chronic disorders, where the microbial or bacterial flora of the bowel is at least one causative or symptom-producing factor, for example, where the microbial or bacterial flora of the bowel manufactures neurotoxins or neurotoxic agents that enter the body through the gastrointestinal (GI) tract, e.g. the colon, and reach the systemic space, e.g., by neural streaming or via the circulation, to reach the central nervous system (CNS), including the brain, the peripheral nervous system (PNS), and other nervous systems; and methods for using them, e.g., for treating, ameliorating and preventing various psychotropic disorders and conditions in mammals. For example, in alternative embodiments, the invention provide compositions and methods for treating, ameliorating and preventing obsessive compulsive disorder group (OCD) psychotropic disorders and conditions, or hepatic encephalopathy (a neuropsychiatric syndrome in patients with either acute or chronic impaired liver function), or a hepatic encephalopathy, or a depressive disorder, a bipolar disorder, an anorexia nervosa, a bulimia, a generalised anxiety disorder, a Tourrets' syndrome, Asperger's syndrome or Attention Deficit Hyperactivity Disorder.

In alternative embodiments, methods and compositions of the invention comprise or comprise use of medications, formulations and pharmaceuticals comprising rifaximin (a semisynthetic derivative of rifamycin) or equivalent active agents that can suppress or eradicate the microbiota super-infection that causes various psychotropic disorders. These compositions have been found to be affective in a broad spectrum of disorders, for example, in the obsessive compulsive disorder group (OCD).

In alternative embodiments, to inhibit or eradicate pathogenic or disease-causing microbiota, e.g., luminal pathogens, a composition (e.g., medication, pharmaceutical or formulation) or method of the invention comprises or comprises use of a rifaximin or a rifaximin in its various molecular versions or polymorphic forms, including for example, the extended intestinal release (EIR) rifaximin, or another rifamycin such as e.g., the rifamycin derivatives rifampicin (or rifampin), rifabutin, rifapentine and/or rifalazil, or XIFAXAN™ (Salix Pharmaceuticals). In alternative embodiments, a composition (e.g., medication, pharmaceutical or formulation) or method of the invention comprises or comprises use of the polymorphic rifaximin in polymorphic form alpha free from other polymorphic forms of rifaximin not derived from form alpha by water absorption or release. Rifaximin is largely a gut-selective antibiotic with very little systemic absorption. It has a broad spectrum activity against both gram-positive and gram-negative pathogens and has good tolerability because of its lack of absorption.

In alternative embodiments, one or more active agents having the same activity as rifaximin, including its properties of being largely a gut-selective antibiotic, having a broad spectrum activity against both gram-positive and gram-negative pathogens, and having with very little systemic absorption, can be used in addition to or as a replacement for rifaximin. In alternative embodiments, one or more active agents having the same activity as rifaximin and having with very little systemic absorption through the gut can be used in addition to or as a replacement for rifaximin. Thus, rifaximin or equivalents thereof can be used in the compositions of the invention or to practice the methods of the invention, as described below.

In alternative embodiments, compositions of the invention (e.g., pharmaceuticals, formulations), and compositions used to practice the methods of the invention, which can comprise rifaximin, polymorphic forms or analogs thereof, or equivalents thereof, are formulated in delivery forms especially applicable to children, and/or for long-term use, particularly for children. In alternative embodiments, compositions of the invention, and compositions used to practice the methods of the invention can take the form of a capsule, a geltab, a pill, a dissolvable wafer, a tablet, a chewable sweet, a lolly (lollypop), a lozenge or a candy, or as a smoothy or a jelly, or ices, ice creams, gelatos, yogurts or drinks.

In alternative embodiments, a delivery form, e.g., a yogurt or a drink, is designed such that the solid component of the rifaximin, polymorphic form or analog thereof, or equivalent thereof, is kept without degradation inside a separate sealed space which can be emptied into the delivery vehicle, e.g., drink or yogurt, in a twist top which will release granules of the active agent (e.g., comprising the rifaximin, polymorphic form or analog thereof, or equivalent thereof, or mixtures thereof) into the drink, or yogurt or the like, and then dissolves and is eaten or drunk by the child.

In alternative embodiments, the compositions of the invention (e.g., pharmaceuticals, formulations), and compositions used to practice the methods of the invention, are enterically coated, e.g., in an MMX enteric extend release format, such that active agent (e.g., comprising the rifaximin, polymorphic form or analog thereof, or equivalent thereof, or mixtures thereof) is delivered throughout the small through to the large bowel to affect its activity on the desired microbe, e.g., the pathogen that causes OCD or related conditions.

In alternative embodiments, a product, a delivery form or formulation of the invention is a flavoured chewable tablet, sweet or candy which the child is directed to take, e.g., more than once a day, e.g., twice or three times daily, to maintain suppression of the OCD producing agents in the flora.

Other forms of administration are also contemplated such as tablets, capsules, liquids, lyophilized forms, creams, ointments, gels, powders and pastes.

### Additional or Optional Ingredients

Compositions and methods of the invention comprise use of rifamycin, molecular versions or polymorphic forms, analogs and rifamycin equivalents thereof, including extended intestinal release (EIR) rifaximin, the rifamycin derivatives rifampicin (or rifampin), rifabutin, rifapentine and rifalazil, or XIFAXAN™, and the like. However, in alternative embodiments, other non-absorbable antibiotics or medications can be used in addition to or in place of rifaximin, or rifampicin or equivalents are used in combination with other antimicrobials or other ingredients.

For example, in alternative embodiment, the invention provides formulations, pharmaceuticals or pharmaceutical preparations comprising at least one active agent, wherein the active agent comprises:
(a) a rifaximin, an extended intestinal release (EIR) rifaximin, a rifamycin derivative, a rifampicin (or rifampin), a rifabutin, a rifapentine, a rifalazil, a bicozamycin, a XIFAXAN™ (Salix Pharmaceuticals), or a mixture or combination thereof,
(b) an aminoglycoside, a gentamycin, a neomycin, a streptomycin, a paromomycin, a verdamicin, a mutamicin, a sisomicin, a netilmicin, a retymicin and/or a kanamycin, or a mixture or combination thereof,
(c) an aztreonam, a AZACTAM™, a CAYSTON™, or a mixture or combination thereof,
(d) a bismuth antibiotic, a bismuth subsalicylate, a PEPTO-BISMOL™, a PINK BISMUTH™, or a mixture or combination thereof,
(f) a mixture or a combination of any one or several of (a), (b), (c) or (d).

In alternative embodiments, rifaximin can also be combined with other active agents or medications in the situation where the causative agent, e.g., the pathogenic microbe, e.g., an OCD pathogen, has some resistance to the rifaximin. For example, in alternative embodiments, rifaximin together with a vancomycin is used.

### Antibiotics, Antimicrobials

In alternative embodiments, other non-absorbable medications can be used in addition to or in place of rifaximin; for example, a vancomycin, a streptomycin, a fidaxomicin, a gentamicin, a kanamycin, an amikacin, an arbekacin, a neomycin, a netilmicin, a paromomycin, rhodostreptomycin, a tobramycin, an apramycin and mixtures thereof. In alternative embodiments, Ampicillin, Sulbactam or Aztreonam macrolides, e.g., Clarithromycin and Azithromycin can also be used. In alternative embodiments, Nitroimidazoles, such as Metronidazole, Tinidazole can also be employed. In alternative embodiments, other anti-clostridial agents such as Ramoplanan can also be use or combined with rifaximin.

In alternative embodiments, doses (unit dosage forms) of rifaximin, polymorphic forms or analogs thereof, or equivalents thereof, or vancomycin, can be from between about 10 mg through or to about 10 gms, or between about 100 mg and 500 mgm, or at 10, 20, 30, 40, 50, 75, 100, 200, 300, 400, 500 or 1000 mgm. Dosages can be adjusted depending on the combination of active agents used, particularly when using two or three or more combination drugs. For example, in alternative embodiments, combination used to practice the compositions or methods of the invention include: streptomycin, Gentamicin, Kanamycin and mixtures thereof; Ampicillin, Sulbactam or Aztreonam and various macrolides, e.g., Clarithromycin; Nitroimidazoles such as Metronidazole; Tinidazole; any anti-clostridial agents such as a Ramoplanan; and/or a Fidaxomicin.

In alternative embodiments, compositions and methods of the invention are used to treat autism, e.g., as described in US patent application 20120252775, which describes methods of treating autism associated with Desulfovibrio overgrowth in the gastrointestinal tract of a patient, but used aztreonam alone or together with a beta-lactamase inhibitor. In alternative embodiments, compositions and methods of the invention comprise use of a rifaximin, or analog or equivalent thereof, with added secondary medications to bolster its activity. In alternative embodiments, compositions and methods of the invention comprise use of rifaximin with vancomycin, or the combination of Rifaximin with Astreonam or Rifaximin with Astreonam together with the beta-lactamase inhibitors. In alternative embodiments these inhibitors are Clavulenic acid, Tazobactam, Sulbactam, LK-157 or equivalents.

In alternative embodiments, compositions and methods of the invention comprise use of a rifaximin, polymorphic forms or analogs thereof, or equivalents thereof combined in double or triple therapy format; including, for example, an astreonam, a streptomycin, a gentamicin, a kanamycin or a macrolides, or any combination thereof. In alternative embodiments metronidazole is also added to ensure that any neural streaming toxins where the bacteria reside in the submucosa, is also treated.

In alternative embodiments, antibiotics and/or other antimicrobials are included in a composition of the invention, e.g., added back to a liquid formulation or preparation of the invention, or cell preparation of the invention. In alternative embodiments, the antimicrobial or antibiotic is or comprises one or more of a: glycopeptide antibiotic, wherein optionally the glycopeptide antibiotic is a vancomycin, a teicoplanin (e.g., TARGOCID™), a telavancin (e.g., VIBATIV™), a bleomycin (e.g., BLENOXANE™), a ramoplanin or a decaplanin; or, a fidaxomycin, a gentamycin, a verdamicin, a mutamicin, a sisomicin, a netilmicin, a retymicin, a neomycin, a streptomycin, a paromomycin, a kanamycin, or an ansamycin, a geldanamycin, an ansamitocin, or an anti-protozoal agent such as nitazoxanide (e.g., DAXON™, DEXIDEX™, KIDONAX™, MITAFAR™, PACOVANTON™, PARAMIX™), a furazolidone (e.g., FUROXONE™, DEPENDAL-M™), a nitroimidazole or metronidazole (e.g., a 5-nitroimidazole, FLAGYL™), a nifuroxazide (e.g., AMBATROL™, ANTINAL™, BACIFURANE™, DIAFURYL™) or a bismuth (e.g., bismuth subsalicylate), also including various groups of antibiotics such as a penicillin (e.g., penicillin G, procaine penicillin, benzathine penicillin or penicillin V), a macrolide (e.g., erythromycin, a clarithromycin (a TRUCLAR™, CRIXAN™, CLARITT™, CLARAC™, BIAXIN™, KLARICID™, KLACID™, KLARAM™, KLABAX™, CLARIPEN™, CLAREM™, CLARIDAR™, FROMILID™, CLACID™, CLACEE™, VIKROL™, INFEX™, CLARIWIN™, RESCLAR™, RANBAXY™ or a CLARIHEXAL™), a dirithromycin (e.g., DYNABAC™), a roxithromycin (e.g., XTHROCIN™, ROXL-150™, ROXO™, SURLID™), a telithromycin (e.g., KETEK™) or an azithromycin such a ZITHROMAX™, AZITHROCIN™), a tetracycline, a cephalosporin, a carbapenem (e.g., imipenem, a meropenem such as MONAN™, MERONEM™), a monobactam, a lincosamide or a clindamycin (e.g., DALACIN™), a quinolone (e.g., a fluoroquinolone) and/or a sulphonamide, a fradicin (e.g., NEOBIOTIC™), or an equivalent thereof or a combination thereof.

In alternative embodiments, the antimicrobial or antibiotic is or comprises one or more of: an aminoglycoside antibiotic (e.g., a gentamycin, a neomycin, a streptomycin, a paromomycin, a verdamicin, a mutamicin, a sisomicin, a netilmicin, a retymicin and/or a kanamycin), amphenicol, ansamycin, beta-lactam (β-lactam), carbapenem, cephalosporin, cephamycin, monobactam, oxacephem, a lincosamide antibiotic (e.g., clindamycin, lincomycin), a macrolide antibiotic (e.g., an azithromycin, clarithromycin, dirithromycin, erythromycin), glycopeptide antibiotic (e.g., a vancomycin, teicoplanin, telavancin, bleomycin, ramoplanin and/or a decaplanin), a polypeptide antibiotic (e.g., actinomycin, such as actinomycin D; bacitracin; bacitracin), tetracycline, or a 2,4-diaminopyrimidine class antibiotic, a clavacin (also known as clairformin, claviform, expansine, clavatin, expansin, gigantin, leucopin, patuline or patulin), or an equivalent thereof or a combination thereof.

### Probiotics and Prebiotics

In alternative embodiments, additives that are also included in a composition of the invention (e.g., a liquid preparation embodiment), or a composition used to practice the invention, includes one or more prebiotics such as inulin, lactulose, extracts of artichoke, chicory root, oats, barley, various legumes, garlic, kale, beans or flacks and at times prebiotics may include herbs.

In alternative embodiments, additives may include flora components such as Bacteroidetes, Firmicutes, Bacillus (e.g., Bacillus thurigiensis) or any combination thereof. In alternative embodiments, cultured components are back to the flora to fortify or expand specific genus or species, e.g., Bacteroidetes, Firmicutes, Bacillus or Bacillus thurigiensis. Probiotics may at times be included as single cultured components. They would avoid multiply cultured components as they lose their implantation characteristics.

### Preservatives, Cryoprotectants, Lyoprotectants

In alternative embodiments, to any composition of the invention (e.g., the liquid preparation embodiment, candies, lollies, drinks and the like) may be added various preservatives, cryoprotectants and/or lyoprotectants, including e.g., various polysaccharides or sugars (such as sucrose, fructose, lactose, mannitol), glycerol, polyethylene glycol (PEG), trehalose, glycine, glucose, dextran and/or erythritol. In alternative embodiments, other cryoprotectants that can be used are ethylene glycol, 1,2-Propanediol, Methylcelliosolve, Dimethyl Formamide, or Dimethylsulphoxide Methanol. In alternative embodiments the content of these cryoprotectants are between about 1% and about 50% but generally between about 5% and about 15% is adequate.

Because of the ability to freeze and/or freeze-dry, or spray dry, any composition of the invention, in alternative embodiments there are different types of final products that can be manufactured. In alternative embodiments a product or formulation of the invention is a liquid. In alternative embodiments a product or formulation of the invention is frozen and kept at e.g. minus 80 degrees for usage later given a cryoprotectant is added.

### Biofilm Disrupting Compounds

In alternative embodiments, biofilm disrupting compounds added into a composition or formulation of the invention (e.g., a liquid preparation embodiment), or used to practice a method of the invention. In alternative embodiments, in practicing the methods of the invention, biofilm disrupting compounds are administered before or during (co-administered), or co-formulated with (e.g., in a multilaminated tablet or capsule), or separately formulated, as the administered composition or formulation of the invention. In alternative embodiments, disrupting biofilms are used to separate from the colonic mucosa an adherent polysaccharide/DNA—containing layer, the so-called “biofilm.

In alternative embodiments, other biofilm disrupting components or agents also can be used, e.g., enzymes such as a deoxyribonuclease (DNase), a N-acetylcysteine, an auranofin, alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7 (a small lytic peptide, see e.g., Kharidia (2011) J. Microbiol. 49(4):663-8, Epub 2011 Sep. 2), Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones and/or macrolide antibiotics or any combination thereof.

In alternative embodiments, biofilm disrupting components or agents are administered before and during the administration of a composition of this invention, e.g., as an FMT, in whatever format or formulation this may take place, for example, as a capsule.

In alternative embodiments, biofilm disrupting agents are added either before treatment and/or during and/or after treatment with a composition of the invention. In alternative embodiments, biofilm disrupting agents are used singly or in combination.

In alternative embodiments, biofilm disrupting agents include particular enzymes and degrading substances including in N-acetylcysteine, deoxyribonuclease (DNase). Others would include Alginate, lyase and Glycoside hydrolase dispersin, Ribonucleic-acid-III inhibiting peptide (RIP), Salvadora persica extracts, Competence-stimulating peptide (CSP) Patulin (PAT) and penicillic acid (PA)/EDTA, Cathelicidin-derived peptides, Small lytic peptide, PTP-7, Nitric oxide, Chlorhexidine, Povidone-iodine (PI), Nanoemulsions, Lytic bacteriophages, Lactoferrin/xylitol hydrogel, Synthetic iron chelators, Cranberry components, Curcumin, Acetyl-11-keto-boswellic acid (AKBA), Barley coffee (BC) components, silver nanoparticles, azithromycin, clarithromycin, gentamicin, streptomycin and also Disodium EDTA. Ozone insufflations of the colon can also be used to disrupt the biofilm.

### Unit Dosage Forms and Formulations, Foods, and Delivery Vehicles

In alternative embodiments, a composition of the invention (e.g., a liquid preparation embodiment) can be further processed by, e.g., spray-drying or equivalent, e.g., spray-drying in an inert gas or freeze-drying under similar conditions, thus ending up with a powdered product. In alternative embodiments, a composition is manufactured, labelled or formulated as a liquid, a suspension, a spray, a gel, a geltab, a semisolid, a tablet, or sachet, a capsule, a lozenge, a chewable or suckable unit dosage form, or any pharmaceutically acceptable formulation or preparation. In alternative embodiments, a composition of the invention is incorporated into a food or a drink (e.g., a yogurt, ice cream, smoothie), a candy, sweet or lolly, or a feed, a nutritional or a food or feed supplement (e.g., liquid, semisolid or solid), and the like.

For example, a composition of the invention can be manufactured, labelled or formulated as an orally disintegrating tablet as described e.g., in U.S. Pat. App. Publication No. 20100297031. A composition of the invention can be a polyol/thickened oil suspension as described in U.S. Pat. No. 6,979,674; 6,245,740. A composition of the invention can be encapsulated, e.g., encapsulated in a glassy matrix as described e.g., in U.S. Pat. App. Publication No. 20100289164; and U.S. Pat. No. 7,799,341. A composition of the invention can be manufactured, labeled or formulated as an excipient particle, e.g., comprising a cellulosic material such as microcrystalline cellulose in intimate association with silicon dioxide, a disintegrant and a polyol, sugar or a polyol/sugar blend as described e.g., in U.S. Pat. App. Publication No. 20100285164. A composition of the invention can be manufactured, labeled or formulated as an orally disintegrating tablet as described e.g., in U.S. Pat. App. Publication No. 20100278930. A composition of the invention can be manufactured, labeled or formulated as a spherical particle, as described e.g., in U.S. Pat. App. Publication No. 20100247665, e.g., comprising a crystalline cellulose and/or powdered cellulose. A composition of the invention can be manufactured, labeled or formulated as a rapidly disintegrating solid preparation useful e.g. as an orally-disintegrating solid preparation, as described e.g., in U.S. Pat. App. Publication No. 20100233278. A composition of the invention can be manufactured, labeled or formulated as a solid preparation for oral application comprising a gum tragacanth and a polyphosphoric acid or salt thereof, as described e.g., in U.S. Pat. App. Publication No. 20100226866.

A composition of the invention can be manufactured, labeled or formulated using a water soluble polyhydroxy compound, hydroxy carboxylic acid and/or polyhydroxy carboxylic acid, as described e.g., in U.S. Pat. App. Publication No. 20100222311. A composition of the invention can be manufactured, labeled or formulated as a lozenge, or a chewable and suckable tablet or other unit dosage form, as described e.g., in U.S. Pat. App. Publication No. 20100184785.

A composition of the invention can be manufactured, labeled or formulated in the form of an agglomerate, as described e.g., in U.S. Pat. App. Publication No. 20100178349. A composition of the invention can be manufactured, labeled or formulated in the form of a gel or paste, as described e.g., in U.S. Pat. App. Publication No. 20060275223. A composition of the invention can be manufactured, labeled or formulated in the form of a soft capsule, as described e.g., in U.S. Pat. Nos. 7,846,475, or 7,763,276.

The polyols used in compositions of the invention can be micronized polyols, e.g., micronized polyols, e.g., as described e.g., in U.S. Pat. App. Publication No. 20100255307, e.g., having a particle size distribution (d₅₀) of from 20 to 60 μm, and a flowability below or equal to 5 s/100 g, or below 5 s/100 g.

### Gradual or Delayed Release Formulations

In alternative embodiments, the invention provides compositions formulated for delayed or gradual enteric release comprising at least one active agent (e.g., a formulation or pharmaceutical preparation of the invention) formulated with a delayed release composition or formulation, coating or encapsulation. In alternative embodiments, formulations or pharmaceutical preparations of the invention are designed or formulated for delivery of active ingredient (e.g., a rifaximin or a rifaximin in its various molecular versions or polymorphic forms, including for example, the extended intestinal release (EIR) rifaximin, or another rifamycin such as e.g., the rifamycin derivatives rifampicin (or rifampin), rifabutin, rifapentine and/or rifalazil, or XIFAXAN™) into the distal small bowel and/or the colon. Thus, for this embodiment, it is important to allow the active ingredient to pass the areas of danger, e.g., stomach acid and pancreatic enzymes and bile, and reach undamaged to be viable in the distal small bowel and especially the colon. In alternative embodiments, a formulation or pharmaceutical preparation of the invention is a liquid formulation, a microbiota-comprising formulation of the invention and/or a frozen or a freeze-dried version thereof. In alternative embodiments, preferably for the encapsulated format, all are in powdered form.

In alternative embodiments, compositions of the invention are formulated for delayed or gradual enteric release using cellulose acetate (CA) and polyethylene glycol (PEG), e.g., as described by Defang et al. (2005) Drug Develop. & Indust. Pharm. 31:677-685, who used CA and PEG with sodium carbonate in a wet granulation production process.

In alternative embodiments, compositions of the invention are formulated for delayed or gradual enteric release using a hydroxypropylmethylcellulose (HPMC), a microcrystalline cellulose (MCC) and magnesium stearate, as described e.g., in Huang et al. (2004) European J. of Pharm. & Biopharm. 58: 607-614).

In alternative embodiments, compositions of the invention are formulated for delayed or gradual enteric release using e.g., a poly(meth)acrylate, e.g. a methacrylic acid copolymer B, a methyl methacrylate and/or a methacrylic acid ester, a polyvinylpyrrolidone (PVP) or a PVP-K90 and a EUDRAGIT® RL PO™, as described e.g., in Kuksal et al. (2006) AAPS Pharm. 7(1), article 1, E1 to E9.

In alternative embodiments, compositions of the invention are formulated for delayed or gradual enteric release as described in U.S. Pat. App. Pub. 20100239667. In alternative embodiments, the composition comprises a solid inner layer sandwiched between two outer layers. The solid inner layer can comprise a formulation or pharmaceutical preparation of the invention and one or more disintegrants and/or exploding agents, one of more effervescent agents or a mixture. Each outer layer can comprise a substantially water soluble and/or crystalline polymer or a mixture of substantially water soluble and/or crystalline polymers, e.g., a polyglycol. These can be adjusted in an exemplary composition of the invention to achieve delivery of the living components of an FMT distally down the bowel.

In alternative embodiments, compositions of the invention are formulated for delayed or gradual enteric release as described in U.S. Pat. App. Pub. 20120183612, which describes stable pharmaceutical formulations comprising active agents in a non-swellable diffusion matrix. In alternative embodiments, a formulation or pharmaceutical preparation of the invention is released from a matrix in a sustained, invariant and, if several active agents are present, independent manner and the matrix is determined with respect to its substantial release characteristics by ethylcellulose and at least one fatty alcohol to deliver bacteria distally.

In alternative embodiments, a formulation or pharmaceutical preparation of the invention is formulated for delayed or gradual enteric release as described in U.S. Pat. No. 6,284,274, which describes a bilayer tablet containing an active agent (e.g., an opiate analgesic), a polyalkylene oxide, a polyvinylpyrrolidone and a lubricant in the first layer and a second osmotic push layer containing polyethylene oxide or carboxy-methylcellulose.

In alternative embodiments, a formulation or pharmaceutical preparation of the invention is formulated for delayed or gradual enteric release as described in U.S. Pat. App. Pub. No. 20030092724, which describes sustained release dosage forms in which a nonopioid analgesic and opioid analgesic are combined in a sustained release layer and in an immediate release layer, sustained release formulations comprising microcrystalline cellulose, EUDRAGIT RSPO™, CAB-O-SIL™, sodium lauryl sulfate, povidone and magnesium stearate.

In alternative embodiments, a formulation or pharmaceutical preparation of the invention is formulated for delayed or gradual enteric release as described in U.S. Pat. App. Pub. 20080299197, describing a multi-layered tablet for a triple combination release of active agents to an environment of use, e.g., in the GI tract. In alternative embodiments, a multi-layered tablet is used, and it can comprise two external drug-containing layers in stacked arrangement with respect to and on opposite sides of an oral dosage form that provides a triple combination release of at least one active agent. In one embodiment the dosage form is an osmotic device, or a gastro-resistant coated core, or a matrix tablet, or a hard capsule. In these alternative embodiments, the external layers may contain biofilm dissolving agents and internal layers the living bacteria.

In alternative embodiments, a formulation or pharmaceutical preparation of the invention is formulated as multiple layer tablet forms, e.g., where a first layer provides an immediate release of a formulation or pharmaceutical preparation of the invention and a second layer provides a controlled-release of another (or the same) formulation or pharmaceutical preparation of the invention, or another active agent, as described e.g., in U.S. Pat. No. 6,514,531 (disclosing a coated trilayer immediate/prolonged release tablet), U.S. Pat. No. 6,087,386 (disclosing a trilayer tablet), U.S. Pat. No. 5,213,807 (disclosing an oral trilayer tablet with a core comprising an active agent and an intermediate coating comprising a substantially impervious/impermeable material to the passage of the first active agent), and U.S. Pat. No. 6,926,907 (disclosing a trilayer tablet that separates a first active agent contained in a film coat from a core comprising a controlled-release second active agent formulated using excipients which control the drug release, the film coat can be an enteric coating configured to delay the release of the active agent until the dosage form reaches an environment where the pH is above four).

In alternative embodiments, a formulation or pharmaceutical preparation of the invention is formulated for delayed or gradual enteric release as described in U.S. Pat. App. Pub. 20120064133, which describes a release-retarding matrix material such as: an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, polyvinylalcohols, polyvinylalcohol copolymers, polyethylene glycols, non-crosslinked polyvinylpyrrolidone, polyvinylacetates, polyvinylacetate copolymers or any combination. In alternative embodiments, spherical pellets are prepared using an extrusion/spheronization technique, of which many are well known in the pharmaceutical art. The pellets can comprise one or more formulations or pharmaceutical preparations of the invention, e.g., the liquid preparation embodiment.

In alternative embodiments, a formulation or pharmaceutical preparation of the invention is formulated for delayed or gradual enteric release as described in U.S. Pat. App. Pub. 20110218216, which describes an extended release pharmaceutical composition for oral administration, and uses a hydrophilic polymer, a hydrophobic material and a hydrophobic polymer or a mixture thereof, with a microenvironment pH modifier. The hydrophobic polymer can be ethylcellulose, cellulose acetate, cellulose propionate, cellulose butyrate, methacrylic acid-acrylic acid copolymers or a mixture thereof. The hydrophilic polymer can be polyvinylpyrrolidone, hydroxypropylcellulose, methylcellulose, hydroxypropylmethyl cellulose, polyethylene oxide, acrylic acid copolymers or a mixture thereof. The hydrophobic material can be a hydrogenated vegetable oil, hydrogenated castor oil, carnauba wax, candellia wax, beeswax, paraffin wax, stearic acid, glyceryl behenate, cetyl alcohol, cetostearyl alcohol or and a mixture thereof. The microenvironment pH modifier can be an inorganic acid, an amino acid, an organic acid or a mixture thereof. Alternatively, the microenvironment pH modifier can be lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid, a salicylic acid, tosylic acid, mesylic acid or malic acid or a mixture thereof.

In alternative embodiments, a formulation or pharmaceutical preparation of the invention is a powder that can be included into a tablet or a suppository. In alternative embodiments, a formulation or pharmaceutical preparation of the invention can be a ‘powder for reconstitution’ as a liquid to be drunk placed down a naso-duodenal tube or used as an enema for patients to take home self-administer enemas for colitis for example. In alternative embodiments, a formulation or pharmaceutical preparation of the invention is micro-encapsulated, formed into tablets and/or placed into capsules, especially enteric-coated capsules. In alternative embodiments, in practicing the methods of the invention, biofilm disrupting compounds are administered before or during (co-administered), or co-formulated with a composition or formulation of the invention. For example, in alternative embodiments, a composition or formulation of the invention and a biofilm disrupting compound (and/or any other alternative component of the invention, as discussed herein) are co-formulated, e.g., as multiple layer tablet form or as a multi-laminated tablet or capsule. In alternative embodiments of methods of the invention, biofilm disrupting compounds are separately formulated.

### Feeds, Drinks, Candies, Nutritional or a Food or Feed Supplements

In alternative embodiments, a formulation or pharmaceutical preparation of the invention is incorporated into a food, a feed, a candy (e.g., a lollypop or a lozenge) a drink, a nutritional or a food or feed supplement (e.g., liquid, semisolid or solid), and the like, as described e.g., in U.S. Pat. App. Publication No. 20100178413. In one embodiment, a formulation or pharmaceutical preparation of the invention is incorporated into (manufactured as) a beverage as described e.g., in U.S. Pat. No. 7,815,956. For example, a composition of the invention is incorporated into a yogurt, an ice cream, a milk or milkshake, a “frosty”, “snow-cone”, or other ice-based mix, and the like.

In alternative embodiments, a formulation or pharmaceutical preparation of the invention is a freeze-dried powder form added to a food, e.g., a yogurt, an ice cream, a milk or milkshake, a “frosty”, “snow-cone”, or other ice-based mix, and the like. In one form of this invention it can be kept in a lid-storage (e.g., of a yogurt or ice cream) such that when it is twisted the powder falls into the product or formulation (e.g., yoghurt or ice cream) and then it can be stirred so as not to have the powder ferment ‘standing on the shelf’. Various flavourings can be added. In alternative embodiments, this is particularly important for administration of a composition of the invention, e.g., a wild type microbiota or a cultured bacteria, to a very young individual and/or a patient with autism or related disease or condition.

In alternative embodiments, these exemplary products are important when administered to children or babies who may have acquired various pathogenic or abnormal bacteria, e.g., E. coli, Clostridia or Disulfovibrio, e.g., as in autism.

### Methods of Use and Applications of Compositions of the Invention

In alternative embodiments, a formulation or pharmaceutical preparation of the invention, and/or a method of the invention, or a use of the invention, is used to treat, ameliorate, prevent or reverse: a neurological disease or syndrome, or a genetically-predisposed or a chronic neurological disorder, where the microbial or bacterial flora of the bowel is at least one causative or symptom-producing factor, for example, where the microbial or bacterial flora of the bowel manufactures neurotoxins or neurotoxic agents that enter the body through the gastrointestinal (GI) tract, e.g. the colon, and reach the systemic space, e.g., by neural streaming or via the circulation, to reach the central nervous system (CNS), including the brain, the peripheral nervous system (PNS), and other nervous systems disorders and conditions. For example, in alternative embodiments, the invention provide compositions and methods for treating, ameliorating and preventing obsessive compulsive disorder group (OCD) psychotropic disorders and conditions, an Attention Deficit Disorder (ADD and ADHD), an obsessive compulsive disorder (OCD), a depression, a schizophrenia and/or a mood disorder, or a hepatic encephalopathy (a neuropsychiatric syndrome in patients with either acute or chronic impaired liver function) or a hepatic encephalopathy, or a depressive disorder, a bipolar disorder, an anorexia nervosa, a bulimia, a generalised anxiety disorder, a Tourrets' syndrome, Asperger's syndrome or Attention Deficit Hyperactivity Disorder.

### Packaging

The invention provides compositions, including preparations, formulations and/or kits, comprising combinations of ingredients, as described herein. In alternative embodiments, these combinations can be mixed and administered together, or alternatively, they can be an individual member of a packaged combination of ingredients, e.g., as manufactured in a separate package, kit or container; or, where all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, the invention also provides a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components (e.g., is a multi-ingredient combination of the invention): a thermoformed “blister” which houses multi-ingredient combination of the invention, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

### Blister Packaging

In alternative embodiments, combinations of ingredients of compositions of the invention, or combinations of ingredients for practicing methods of the invention, can be packaged alone or in combinations, e.g., as “blister packages” or as a plurality of packettes, including as lidded blister packages, lidded blister or blister card or packets or packettes, or a shrink wrap.

In alternative embodiments, laminated aluminium foil blister packs are used, e.g., for the preparation of drugs designed to dissolve immediately in the mouth of a patient. This exemplary process comprises having the drug combinations of the invention prepared as an aqueous solution(s) which are dispensed (e.g., by measured dose) into an aluminium (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminium (e.g., alufoil) lidding material. In one aspect, hermetically-sealed high barrier aluminium (e.g., alufoil) laminates are used. In one aspect, any of the invention's products of manufacture, including kits or blister packs, use foil laminations and strip packs, stick packs, sachets and pouches, peelable and non-peelable laminations combining foil, paper, and film for high barrier packaging.

In alternative embodiments, any of the invention's multi-ingredient combinations or products of manufacture, including kits or blister packs, include memory aids to help remind patients when and how to take the drug. This safeguards the drug's efficacy by protecting each tablet, geltab or pill until it's taken; gives the product or kit portability, makes it easy to take a dose anytime or anywhere.

The invention will be further described with reference to the following examples; however, it is to be understood that the invention is not limited to such examples.

### EXAMPLES

### Example 1

### Exemplary Treatment of OCD in Adult With Rifaximin

A 43 year old female married office worker had suffered with OCD since childhood. Having been treated with various pharmacologic agents through life her behaviour patterns were largely controlled except for one behaviour of needing to check gas outlets and powerpoints every night in case her children were hurt or damaged overnight by some unforseen accident. This continued to be a ‘sleep-draining’ issue not controllable by Zoloft, anfranil and other drugs.

She was commenced on rifaximin 500 mg capsules twice daily for the first week, then 1 g twice daily thereafter.

Within 10 days she noticed an absence of the need to check the gas outlets and powerpoints and was able to retire sleep through the night, perhaps first time in many years. This improvement was almost universal for the next 3 months of monitoring her behaviour. She remains on long term rifaximin.

### Example 2

### Exemplary Treatment of OCD of Child With Rifaximin and Vancomycin

A 13 year old male child was completely well until an overseas trip and a diarrhoeal illness which seemed to last 2-3 weeks. Seemingly unrelated he developed rather quickly, over several months patterns of progressively severe, repetitive behavioural changes diagnosed as OCD. This was managed by pharmacologic agents in a leading Institute for Anxiety Disorders. Drugs included fluoxetine and escitalopram. He had a documented number of some 56 repetitive behavioural patterns documented by the Institute.

He was commenced on increasing dose of vancomycin 250 mg bid and rifaxamin 550 mg bid. After four weeks of treatment there was already an improvement with a measurable reduction of the OCD pattern. From being unable to attend school nor see his school peers he was able to invite friends over as the symptoms were much less intrusive.

The dosage of the medications were raised to 500 mg bid of vancomycin and 2.2 g of rifaxamin per day in a bid dose. Over the next 12 months of treatment the repetitive behavioural groupings were reduced to 2 per day and he was able to cut down on his pharmacological treatment. He returned to school where he again reached the same level of excellence prior to developing his OCD syndrome.

He continues on the same dosages of vancomycin and rifaxamin 22 months later. Fluoxetine has now been stopped.

### Example 3

A 34 month old child who was diagnosed with development of Autistic Spectrum Disorder (ASD) with change in bowel habit, withdrawal from ‘society’ and loss of vocabulary/words/vocalizing ability as well as reduced socialisation and eye contact was treated with Aztreonam 50 mg 3 times daily. About two weeks after starting the Aztreonam the parents noticed ‘greater socialisation’ with the other children and reduction in the loose motions which initially alternated with hard. There was greater eye contact but at this stage no increase in word power. 125 mg tds of Vancomycin was added and the bowel function improved to a very large volume output compared with originally so that presumed ‘constipation’ was being handled. What is more relevant is that the child appeared to regain its previous language skills picking up at least five more words than before over 3 months. There was less repetitive movement than before and even further eye contact. The treatment continued for three more months and the improvement although fluctuating, continued as well.

### Example 4

Twin sisters who were diagnosed with Autism Spectrum Disorder (ASD) at different levels of reading capability, writing and comprehension in Grade 3 and 4, together with abdominal cramping and alternating hard/soft defecation changes were started on oral Gentamicin 80 mg 3 times daily in capsules. Each had a component of OCD in her psychotropic disorder but within three weeks the use of the Gentamicin had helped the OCD to becoming less noticed with marked reduction in the number of episodes of OCD behaviour. After three weeks Rifaximin was added to the mix of drugs at a dose of 500 mg twice daily and the patients were further observed with a combination of Gentamicin and Rifaximin. Further improvement occurred with vocabulary increase when the patient was next reviewed at three months. There was some lessening in the stool quality but overall the psychotropic improvement was quite marked.

### Example 5

A seven year old male patient was brought by his father for consultation. He had been suffering from ASD since early childhood but also had chronic constipation and abdominal discomfort. The treatment was focussed on the constipation problem as this was the major component of his complaints when interviewed. He was commenced on to 50 mg Vancomycin twice daily for the first week increasing to 250 mg 3 times daily as an oral dose which is not absorbed. Initially there was some increase in abdominal discomfort as wind increased and gurgling increased and it was felt that the bowel was attempting to empty. Within a few days however the bowel function improved from once every three to four days to daily or twice daily and even three times daily evacuation, and the pain progressively disappeared. At four week interview the constipation and the pain were markedly improved and the parents were both happy with the result. However they also noticed a change in behaviour of the child. He was much calmer, with his low vocabulary and inability to complain formally, he was no longer screaming and vocalising his pain using repetitive movements. In fact they came down quite markedly and he was able to cuddle and actually show a different attitude towards his sister. He showed concern when the sister hurt her arm which had previously not been noticed i.e. that he would show concern for someone else's suffering. After four months it was noted that there was better communication between the parents and the boy. This is both with verbal and non-verbal communication. The patient remains on treatment for nine months, sometimes varying between 750 mg and 1 g of Vancomycin orally without any undue side effects from the medication.

A number of embodiments of the invention have been described. Nevertheless, it will be understood that various modifications may be made without departing from the spirit and scope of the invention. Accordingly, other embodiments are within the scope of the following claims.

1. A method for the treating, ameliorating and preventing obsessive compulsive disorder group (OCD) psychotropic disorders and conditions, an Attention Deficit Disorder (ADD and ADHD), an obsessive compulsive disorder (OCD), an anorexia nervosa, a bulimia, a generalised anxiety disorder, a Tourrets' syndrome, Asperger's syndrome or Attention Deficit Hyperactivity Disorder, comprising administering to an individual in need thereof:
a formulation, a pharmaceutical or a pharmaceutical preparation comprising at least one active agent, wherein the active agent comprises:
(a) a rifaximin, an extended intestinal release (EIR) rifaximin, a rifamycin derivative, a rifampicin (or rifampin), a rifabutin, a rifapentine, a rifalazil, a bicozamycin, or a mixture or combination thereof,
(b) an aminoglycoside, a gentamycin, a neomycin, a streptomycin, a paromomycin, a verdamicin, a mutamicin, a sisomicin, a netilmicin, a retymicin and/or a kanamycin, or a mixture or combination thereof,
(c) an aztreonam, an aztreonam macrolide, a clarithromycin, a dirithromycin, a roxithromycin, a telithromycin, an azithromycin, or a mixture or combination thereof,
(d) a bismuth antibiotic, a bismuth subsalicylate, or a mixture or combination thereof,
(e) a vancomycin, a streptomycin, a fidaxomicin, a gentamicin, a kanamycin, an amikacin, an arbekacin, a neomycin, a netilmicin, a paromomycin, rhodostreptomycin, a tobramycin, an apramycin, or a mixture or combination thereof, or
(f) a mixture or a combination of any one or several of (a), (b), (c), (d) or (e).

2. The method of claim 1 wherein the formulation, the pharmaceutical or the pharmaceutical preparation is formulated as a chewable delivery vehicle, a gum, a gummy, a candy, a lozenge, an ice cream or an ice, or a yogurt.

3. The method of claim 1 wherein a unit dosage is a pediatric unit dosage, and optionally the unit dosage is between about 10 mg and 1100 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000 or 1100 or more mg per unit dose.

4. The method of claim 1 wherein a daily dosage is about 100, 125, 150, 175, 200, 225, 250, 275, 300, 325, 350, 375, 400, 425, 450, 475, 500, 600, 700, 750, 800, 900, 1000 or 1100 or more mg per day, or between about 100 and 1100 mgm per day.

5. The method of claim 1 wherein a unit dosage is set for bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.

6. The method of claim 1 wherein the formulation, pharmaceutical or pharmaceutical preparation comprises a combination of a rifaximin together with a vancomycin.

7. The method of claim 1 wherein the formulation, the pharmaceutical or the pharmaceutical preparation further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.

8. The method of claim 1 wherein the formulation, the pharmaceutical or the pharmaceutical preparation further comprises a preservative, a benzoic acid or a potassium sorbate.

9. The method of claim 1 wherein the formulation, the pharmaceutical or the pharmaceutical preparation further comprises, or has added to: at least one probiotic or prebiotic,
wherein optionally the prebiotic comprises an inulin, lactulose, extracts of artichoke, chicory root, oats, barley, various legumes, garlic, kale, beans or flacks or an herb,
wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E coli, a Strep fecalis and equivalents.

10. The method of claim 1 wherein the formulation, the pharmaceutical or the pharmaceutical preparation further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.

11. The method of claim 1 wherein the formulation, the pharmaceutical or the pharmaceutical preparation further comprises, or has added to: at least one an anti-inflammatory agent, wherein optionally the inflammatory agent comprises or is a 4 or a 5-amino-salicylate, an olsalazine, a mesalazine (also known as mesalamine or a 5-aminosalicylic acid (5-ASA), a sulfasalazine and/or a balsalazide, or an equivalent thereof or a combination thereof.

12. The method of claim 1 wherein the formulation, the pharmaceutical or the pharmaceutical preparation further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.

13. The method of claim 1 wherein the formulation, the pharmaceutical or the pharmaceutical preparation further comprises, or has added to: at least one Biofilm Disrupting Compound,
wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

14. The method of claim 1 wherein the formulation, the pharmaceutical or the pharmaceutical preparation is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.

15. The method of claim 1 wherein the formulation, the pharmaceutical or the pharmaceutical preparation further comprises an additional antimicrobial or antibiotic,
wherein optionally the additional antimicrobial or antibiotic comprises:
an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, an anti-clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
an equivalent thereof or a combination thereof.

16. The method of claim 1 wherein the formulation, the pharmaceutical or the pharmaceutical preparation is contained in a delivery vehicle, product of manufacture, container, syringe, device or bag.

17. The method of claim 1, wherein the formulation, the pharmaceutical or the pharmaceutical preparation is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

18. A formulation, a pharmaceutical or a pharmaceutical preparation comprising at least one active agent, wherein the active agent comprises:
(a) a rifaximin, an extended intestinal release (EIR) rifaximin, a rifamycin derivative, a rifampicin (or rifampin), a rifabutin, a rifapentine, a rifalazil, a bicozamycin, or a mixture or combination thereof,
(b) an aminoglycoside, a gentamycin, a neomycin, a streptomycin, a paromomycin, a verdamicin, a mutamicin, a sisomicin, a netilmicin, a retymicin and/or a kanamycin, or a mixture or combination thereof,
(c) an aztreonam, an aztreonam macrolide, a clarithromycin, a dirithromycin, a roxithromycin, a telithromycin, an azithromycin, or a mixture or combination thereof,
(d) a bismuth antibiotic, a bismuth subsalicylate, or a mixture or combination thereof,
(e) a vancomycin, a streptomycin, a fidaxomicin, a gentamicin, a kanamycin, an amikacin, an arbekacin, a neomycin, a netilmicin, a paromomycin, rhodostreptomycin, a tobramycin, an apramycin, or a mixture or combination thereof,
(f) a mixture or a combination of any one or several of (a), (b), (c), (d) or (e); or
in the preparation of a medicament for treating, ameliorating and preventing obsessive compulsive disorder group (OCD) psychotropic disorders and conditions, an Attention Deficit Disorder (ADD and ADHD), an obsessive compulsive disorder (OCD), an anorexia nervosa, a bulimia, a generalised anxiety disorder, a Tourrets' syndrome, Asperger's syndrome or Attention Deficit Hyperactivity Disorder.

19. A formulation, a pharmaceutical or a pharmaceutical preparation comprising at least three active agents, wherein the active agent comprises:
(a) a rifaximin, an extended intestinal release (EIR) rifaximin, a rifamycin derivative, a rifampicin or rifampin, a rifabutin, a rifapentine, a rifalazil, a bicozamycin, or a mixture or combination thereof,
(b) an aztreonam, an aztreonam macrolide, a clarithromycin, a dirithromycin, a roxithromycin, a telithromycin, an azithromycin, or a mixture or combination thereof, and
(c) a tetracycline.

20. A formulation, a pharmaceutical or a pharmaceutical preparation comprising a rifabutin, a roxithromycin and a tetracycline.
